SEQUENCE LISTING (11 sequences)

SEQ ID NO: 1 - protein, 673 aa, Homo sapiens
  PDE4D3 (1)..(673)
  LF1_splice_site (17)..(17)
  UCR1_site_-13 (37)..(37)
  UCR1_start (50)..(50)
  Ser54 (54)..(54)
  Ser579 (579)..(579)
MMHVNNFPFRRHSWICFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 2 - protein, 664 aa, Homo sapiens
  PDE4D_core (1)..(664)
  LF1_splice_site (2)..(2)
  UCR1_site_-13 (22)..(22)
  UCR1_site (35)..(35)
  SER54 (39)..(39)
  SER579 (564)..(564)
MFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDTHHHHHH

SEQ ID NO: 3 - protein, 747 aa, Homo sapiens
  PDE4D5 (1)..(747)
  isoform_specific_N-terminus (1)..(90)
  LF1_splice_site (91)..(91)
  UCR1_site-13 (111)..(111)
  UCR1_site (124)..(124)
  Ser54 (128)..(128)
  Ser579 (653)..(653)
MAQQTSPDTLTVPEVDNPHCPNPWLNEDLVKSLRENLLQHEKSKTARKSVSPKLSPVISPRNSPRLLRRMLLSSNIPKQRRFTVAHTCKLFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 4 - protein, 689 aa, Homo sapiens
  PDE4D6 (1)..(689)
  isoform_specific_N-terminus (1)..(32)
  LF1_splice_site (33)..(33)
  UCR1_start-13 (53)..(53)
  UCR1_start (66)..(66)
  Ser54 (70)..(70)
  Ser579 (595)..(595)
MAFVWDPLGATVPGPSTRAKSRLRFSKSYSKLFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 5 - protein, 750 aa, Homo sapiens
  PDE4D7 (1)..(750)
  isoform_specific_N-terminus (1)..(93)
  LF1_splice_site (94)..(94)
  UCR1_start-13 (114)..(114)
  UCR1_start (127)..(127)
  Ser54 (131)..(131)
  Ser579 (656)..(656)
MKRNTCDLLSRSKSASEETLHSSNEEEDPFRGMEPYLVRRLSCRNIQLPPLAFRQLEQADLKSESENIQRPTSLPLKILPLIAITSAESSGKLFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 6 - protein, 679 aa, Homo sapiens
  PDE4D8 (1)..(679)
  isoform_specific_N-terminus (1)..(23)
  LF1_splice_site (23)..(23)
  UCR1_start-13 (43)..(43)
  UCR1_start (56)..(56)
  Ser54 (60)..(60)
  Ser579 (585)..(585)
MSIIMKPRSRSTSSLRTAEAKLFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 7 - protein, 809 aa, Homo sapiens
  PDE4D4 (1)..(809)
  LF1_splice_site (153)..(153)
  UCR1_start_-13 (173)..(173)
  UCR_1_start (186)..(186)
  Ser54 (190)..(190)
  Ser579 (715)..(715)
MEAEGSSAPARAGSGEGSDSAGGATLKAPKHLWRHEQHHQYPLRQPQFRLLHPHHHLPPPPPPSPQPQPQCPLQPPPPPPLPPPPPPPGAARGRYASSGATGRVRHRGYSDTERYLYCRAMDRTSYAVETGHRPGLKKSRMSWPSSFQGLRRFDVDNGTSAGRSPLDPMTSPGSGLILQANFVHSQRRESFLYRSDSDYDLSPKSMSRNSSIASDIHGDDLIVTPFAQVLASLRTVRNNFAALTNLQDRAPSKRSPMCNQPSINKATITEEAYQKLASETLEELDWCLDQLETLQTRHSVSEMASNKFKRMLNRELTHLSEMSRSGNQVSEFISNTFLDKQHEVEIPSPTQKEKEKKKRPMSQISGVKKLMHSSSLTNSSIPRFGVKTEQEDVLAKELEDVNKWGLHVFRIAELSGNRPLTVIMHTIFQERDLLKTFKIPVDTLITYLMTLEDHYHADVAYHNNIHAADVVQSTHVLLSTPALEAVFTDLEILAAIFASAIHDVDHPGVSNQFLINTNSELALMYNDSSVLENHHLAVGFKLLQEENCDIFQNLTKKQRQSLRKMVIDIVLATDMSKHMNLLADLKTMVETKKVTSSGVLLLDNYSDRIQVLQNMVHCADLSNPTKPLQLYRQWTDRIMEEFFRQGDRERERGMEISPMCDKHNASVEKSQVGFIDYIVHPLWETWADLVHPDAQDILDTLEDNREWYQSTIPQSPSPAPDDPEEGRQGQTEKFQFELTLEEDGESDTEKDSGSQVEEDTSCSDSKTLCTQDSESTEIPLDEQVEEEAVGEEEESQPEACVIDDRSPDT

SEQ ID NO: 8 - DNA, 1974 bp, Homo sapiens
  PDE4D3_coding_sequence (1)..(1974)
tttgatgtggacaatggcacatctgcgggacggagtcccttggatcccatgaccagcccaggatccgggctaattctccaagcaaattttgtccacagtcaacgacgggagtccttcctgtatcgatccgacagcgattatgacctctctccaaagtctatgtcccggaactcctccattgccagtgatatacacggagatgacttgattgtgactccatttgctcaggtcttggccagtctgcgaactgtacgaaacaactttgctgcattaactaatttgcaagatcgagcacctagcaaaagatcacccatgtgcaaccaaccatccatcaacaaagccaccataacagaggaggcctaccagaaactggccagcgagaccctggaggagctggactggtgtctggaccagctagagaccctacagaccaggcactccgtcagtgagatggcctccaacaagtttaaaaggatgcttaatcgggagctcacccatctctctgaaatgagtcggtctggaaatcaagtgtcagagtttatatcaaacacattcttagataagcaacatgaagtggaaattccttctccaactcagaaggaaaaggagaaaaagaaaagaccaatgtctcagatcagtggagtcaagaaattgatgcacagctctagtctgactaattcaagtatcccaaggtttggagttaaaactgaacaagaagatgtccttgccaaggaactagaagatgtgaacaaatggggtcttcatgttttcagaatagcagagttgtctggtaaccggcccttgactgttatcatgcacaccatttttcaggaacgggatttattaaaaacatttaaaattccagtagatactttaattacatatcttatgactctcgaagaccattaccatgctgatgtggcctatcacaacaatatccatgctgcagatgttgtccagtctactcatgtgctattatctacacctgctttggaggctgtgtttacagatttggagattcttgcagcaatttttgccagtgcaatacatgatgtagatcatcctggtgtgtccaatcaatttctgatcaatacaaactctgaacttgccttgatgtacaatgattcctcagtcttagagaaccatcatttggctgtgggctttaaattgcttcaggaagaaaactgtgacattttccagaatttgaccaaaaaacaaagacaatctttaaggaaaatggtcattgacatcgtacttgcaacagatatgtcaaaacacatgaatctactggctgatttgaagactatggttgaaactaagaaagtgacaagctctggagttcttcttcttgataattattccgataggattcaggttcttcagaatatggtgcactgtgcagatctgagcaacccaacaaagcctctccagctgtaccgccagtggacggaccggataatggaggagttcttccgccaaggagaccgagagagggaacgtggcatggagataagccccatgtgtgacaagcacaatgcttccgtggaaaaatcacaggtgggcttcatagactatattgttcatcccctctgggagacatgggcagacctcgtccaccctgacgcccaggatattttggacactttggaggacaatcgtgaatggtaccagagcacaatccctcagagcccctctcctgcacctgatgacccagaggagggccggcagggtcaaactgagaaattccagtttgaactaactttagaggaagatggtgagtcagacacggaaaaggacagtggcagtcaagtggaagaagacactagctgcagtgactccaagactctttgtactcaagactcagagtctactgaaattccccttgatgaacaggttgaagaggaggcagtaggggaagaagaggaaagccagcctgaagcctgtgtcatagatgatcgttctcctgacacgtaa

SEQ ID NO: 9 - DNA, 36 bp, Artificial
  PCR primer
gatgaattcaagctttttgatgtggacaatggcaca

SEQ ID NO: 10 - DNA, 45 bp, Artificial
  PCR primer
gtgatatctcattatcacgtgtcaggagaacgatcatctatgaca

SEQ ID NO: 11 - DNA, 61 bp, Artificial
  PCR primer
gtgatatctcattatcaatgggatggtgatggtgcgtgtcaggagaacgatcatctatgac

(19) United States
(12) Patent Application Publication
(10) Pub. No.: US 2005/0106581 A1
(43) Pub. Date: May 19, 2005

(54) Modified phosphodiesterase polypeptides with altered physiochemical properties

(76) Inventors: Ulrich Certa, Allschwil (CH); Juergen Fingerle, Rheinfelden (DE); Peter Nelboeck-Hochstetter, Basel (CH); Hans-Joachim Schoenfeld, Freiburg (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 10/735,973

(22) Filed: Dec. 15, 2003

(30) Foreign Application Priority Data
Dec. 17, 2002 (EP) 02028057.4

(51) Int. Cl. C12Q 1/68; C07H 21/04; C12N 9/16

(52) U.S. Cl. 435/6; 435/69.1; 435/196; 435/320.1; 435/325; 536/23.2

(57) ABSTRACT

Modified phosphodiesterase (PDE) polypeptides with altered physicochemical properties are disclosed, together with the polynucleotides encoding such modified PDE polypeptides, antibodies against such polypeptides and methods of preparation. Such polypeptides are useful for screening assays for identification of agonists or antagonists of phosphodiesterase activity.

The invention relates to modified phosphodiesterase (PDE) polypeptides with altered physicochemical properties which are particularly useful in screening assays for the identification of agonists or antagonists of PDEs, the polynucleotides encoding such modified PDE polypeptides, antibodies against such polypeptides, methods of preparation and uses of such polypeptides.

### BACKGROUND OF THE INVENTION

The phosphodiesterases (PDEs) represent a family of enzymes that catalyze the hydrolysis of the various cyclic nucleoside monophosphates (including cAMP). These cyclic nucleotides have been found to act as second messengers within the cells by carrying impulses from cell surface receptors having bound various hormones and neurotransmitters to the inside of the cells. The task of phosphodiesterases is to degrade these cyclic mononucleotides once their messenger role is completed thereby regulating the level of cyclic nucleotides within the cells and maintaining cyclic nucleotide homeostasis.

Ten families of PDEs have been identified, designated PDE1 to PDE10. Within each family there are two or more related but distinct gene products (A, B, C, etc.) and for each of these alternative mRNA processing gives rise to multiple splice variants, identified by an additional arabic numeral in accordance with the most recent nomenclature recommendation (Molecular Pharmacology 46, 399-405, 1994).

All PDE gene products identified so far have one catalytic and a least one regulatory domain. The catalytic domain lies towards the carboxylic acid terminus of each PDE protein and has the greatest homology between the PDE families, being >75% homologous at the amino acid level (Perry and Higgs, Curr. Opinion Chem. Biol. 2, 472-481, 1998). Nevertheless, each of the more than 30 known PDEs have individually distinct substrate specificities, kinetic characteristics, regulatory properties and cellular and subcellular distributions (Houslay and Milligan, Trends in Biochem. Sci. 22, 217-224, 1997).

PDEs 4, 7 and 8 are highly specific for cAMP. PDEs 5, 6, 9 and 10 are selective for cGMP. PDE3s bind cAMP and cGMP with similar affinity, but hydrolyse cAMP most efficiently, cGMP rather poorly. PDE3s are, therefore, negatively regulated in their cAMP hydrolysing ability by cGMP. PDEs 1 and 2 hydrolyse both cAMP and cGMP, but with PDE1 the relative efficiencies vary with isoenzyme subtype (Perry and Higgs, Curr. Opinion Chem. Biol. 2, 472-481, 1998).

The amino terminal ends of PDEs consist of the regulatory domains, which are very different both between families and between variants within families. This region contains variously: a binding domain for Ca2+-calmodulin (CaM) in PDE1; non-catalytic cGMP-35 binding sites in PDEs 2, 5 and 6; a binding domain for the signalling G-protein transducin in PDE6. The amino terminal region also contains protein- and membrane-targeting sequences in several PDE3s and PDE4s, as well as protein kinase phosphorylation sites in PDEs 1, 3, 4 and 5. These phosphorylation sites are likely to be important in regulation of catalytic activity and/or subcellular location (Perry and Higgs, Curr. Opinion Chem. Biol. 2, 472-481, 1998).

Of particular interest to this invention are the PDE4 enzymes. PDE4 enzymes share a common structure, as deduced from their amino acid sequences (Beavo and Reifsnyder, Trends Pharmacol. Sci. 11, 150-155, 1990; Bolger at al., Mol. Cell Biol. 13, 6558-6571, 1993; Houslay, Sullivan and Bolger, Adv. Pharmacol. 44, 225-242, 1998). Members of each gene family (PDE4A, PDE4B, PDE4C, PDE4D) share common C-terminal regions, different for each family, and catalytic domains that for all PDE4 isoforms are very similar (84% homology over about 360 amino acids across all PDE4s; Houslay, Sullivan and Bolger, Adv. Pharmacol. 44, 225-242, 1998).

From N-terminus to catalytic region, the sequence in “long form” PDE4s can be divided into 5 regions, three of which are isoform-specific (N-terminal region, linker regions 1 and 2, or LR1 and LR2) and two, more conserved regions, that are broadly similar between all isoforms, the upstream conserved regions 1 and 2 (UCR1 and UCR2). “Short form” PDE4s, e.g. PDE4A1, PDE4B2, PDE4D1, PDE4D2, lack UCR1 and LR1 plus differing amounts of the N-terminal region of UCR2. Throughout all regions are potential phosphorylation sites for a variety of kinases, including PKA (e.g. Ser 54 in human PDE4D3), nitrogen activated protein kinases (e.g. Ser 487 of human PDE4B2), casein kinase II (e.g. Ser 489 of PDE4B2) and calcium-diacylglycerol dependent protein kinases (Houslay, Sullivan and Bolger, Adv. Pharmacol. 44, 225-242, 1998). Phosphorylations at some of these sites have been shown to activate the PDEs (e.g. Ser 54), others serve to inhibit. There is also evidence that some phosphorylations serve to prime the enzymes ready for subsequent activation by further phosphorylation at a different site or sites (Houslay, Sullivan and Bolger, Adv. Pharmacol. 44, 225-242, 1998). Other auto-regulatory sites may be found in the N-terminal sequence of certain PDE4s (Bolger at al., Mol. Cell Biol. 13, 6558-6571, 1993, McPhee et al., Biochem. J. 310, 965-974, 1995).

Specific PDE4 inhibition may be useful over a very wide range of disease areas. These include: asthma, a topic dermatitis, depression, reperfusion injury, septic shock, toxic shock, autoimmune diabetes, AIDS, Crohn's disease, multiple sclerosis, cerebral ischemia, psoriasis, allograft rejection, restenosis, stroke, ulcerative colitis, cachexia, cerebral malaria, allergic rhinoconjunctivitis, osteoarthritis, rheutmatoid arthritis, autoimmune encephalomyelitis (Houslay, Sullivan and Bolger, Adv. Pharmacol. 44, 225-242, 1998, WO02/074992). In the area of asthma, PDE4 inhibition helps to increase cAMP in bronchial smooth muscle, thereby producing a modest bronchodilatory effect, of use in the alleviation of asthmatic symptoms. But perhaps most importantly, inhibition of PDE4s is now a recognised method by which to suppress immune and inflammatory cell responses (Hughes et al. Drug Discov. Today 2, 89-101, 1997; Torphy, Am. J. Respir. Crit. Care Med. 157, 351-370, 1998; Teixeira et al., Trends Pharm. Sci. 18, 164-171, 1997).

PDE4s play major roles in modulating the activity of virtually every cell type involved in the inflammatory process. Immune and inflammatory conditions occur when recruitment of leukocytes from the blood compartment into tissues is either uncontrolled, inappropriate, prolonged or directed against self In asthma, rheumatoid arthritis and multiple sclerosis, infiltration of tissues with inflammatory cells is prolonged and intense, leading ultimately to severe (and self-perpetuating) damage and loss of function. Acute disregulation of the immune system occurs in such conditions as acute respiratory distress syndrome (ARDS) where an overwhelming and generalised inflammatory response can frequently lead to death. There is also substantial evidence which suggests that inflammation may play a part in defining the extent of injury resulting from reperfusion following ischaemia, at least in brain and lung (Entman and Smith, Cardiovasc. Res. 28, 1301-1311, 1994).

The PDE4D phosphodiesterases, which are the most preferred focus of the present invention, exist in mammals in the form of isoenzymes (which represent different molecular forms of the same enzyme polypeptide). These phosphodiesterases are localized in the cytosol of the cell and are unassociated with any known membraneous structures. The PDE4D isoenzymes specifically degrade cAMP and are a common target for such pharmacological agents as antidepressants (for example, rolipram). Several splice forms of PDE4D are known. The short forms, PDE4D1 and PDE4D2, lack UCR1 and LR1 sites. Rather than these, interest of the present invention focuses on the long isoforms, of which 6 are known, namely PDE4D3, PDE4D4, PDE4D5, PDE4D6, PDE4D7 and PDE4D8. All of these have in common the LR1 and UCR1 sites and the domains located at the C-terminus of these sites, but they have different N-terminal domains. Isoforms PDE4D6, D7 and D8 were recently disclosed in WO02/074992.

For identification of compounds that inhibit PDE4D isoenzymes, screening assays are used that are either whole cell assays in which the cells are treated with the inhibitor and cAMP hydrolysis is measured, or in vitro assays which use purified PDE4D isoenzymes either isolated from their natural sources or from cells overexpressing recombinant PDE4D isoenzymes. Such assays are described e.g. in WO00/09504, WO01/35979, WO01/68600 and WO98/45268. WO01/35979 describes the purification of recombinant PDE4D expressed in Saccharomyces Cerevisiae. The use of recombinant native forms of PDE4D for the identification of PDE agonists or antagonists has the disadvantage that the PDE4D poypeptide does not have optimal physicochemical characteristics, especially with respect to aggregate formation. Aggregate formation can lead to protein precipitation and is known to potentially decrease the activity of enzymes, compared to the authentic oligomeric molecules. Thus, aggregation of the native forms of PDE4D polypeptides in screening assays aimed at identifying agonists or antagonists of PDE4D phosphodiesterase activity negatively affects the quality of these assays. In addition, the use of assays based on individual recombinant PDE4D isoforms has the disadvantage of potentially resulting in the identification of agonists or antagonists that are specific for a single isoform of a PDE4D only, instead of agonists or antagonists which act on all long or short form PDE4D isoforms together.

### BRIEF DESCRIPTION OF THE DRAWINGS

FIG. 1: Specific phosphodiesterase activity for the different PDE4D isoforms D3 and D5 to D8, and the PDE4D core construct (DC).

FIG. 2: Aggregate formation by different PDE4D isoforms and the core construct. In this figure, the gel filtration profiles of different isoforms of PDE4D (D3, D5, D6, D7, D8), and the core construct (DC) are shown. The first elution peak represents PDE4D aggregates. The pronounced shoulder in the elution profile of DC, where polypeptides of approx. 300,000 Dalton elute, indicates a decrease in aggregation of DC, as compared to the five isoforms of PDE4D.

FIG. 3: Quantitation of aggregation of the core construct (DC) of PDE4D, and of the tubulin content of the core construct preparations. In A), the elution profile of DC is shown, with the fractions indicated on the bottom. B) shows an SDS-PAGE of the different fractions from the gel filtration. C) shows the analysis of optical densities of individual fractions corresponding to the Coomassie stained SDS-PAGE in B).

FIG. 4: Differences in aggregation and tubulin content of different isoforms and the core construct of PDE. A) SDS-PAGE gels are shown for DC and five isoforms of PDE4D which were analysed as described above. B) The percentage of non-aggregated PDE4D is shown for the core construct and the isoforms D3 and D5 to D8, based on total PDE4D protein. DC has the highest percentage of non-aggregated PDE4D, as compared to the five isoforms. C) This figure shows the ratio between PDE4D and tubulin for DC and the isoforms D3 and D5 to D8, based on total PDE mass and total tubulin mass. As can be seen, the tubulin content of DC is significantly lower than that of the five PDE4D isoforms indicated.

FIG. 5: Activity assay. Phosphodiesterase activity is shown both for the PDE4D core construct (DC) and PDE4D3 (D3).

FIG. 6: Rolipram inhibition. Phosphodiesterase activity of the PDE4D core construct (DC) can be inhibited by Rolipram. The IC50 of DC phosphodiesterase activity by Rolipram inhibition was 0.34±0.06 mM.

### DETAILED DESCRIPTION OF THE INVENITON

The term “polypeptide” as used herein, refers to a polymer of amino acids, and not to a specific length. Thus, peptides, oligopeptides and proteins are included within the definition of polypeptide.

The term “physicochemical properties” as used herein refers to the propensity of polypeptides to monomer formation, oligomer formation or aggregate formation.

The terms “aggregation” and “aggregate formation” as used herein are used interchangeably and refer to the type of association of polypeptide chains which, in contrast to oligomer formation, is an irreversible process and does not follow the law of mass interaction. Aggregation progresses in a time dependent manner and does not stop when a defined number of chains have associated. Aggregation of polypeptides can lead to protein precipitation because the molecular size of the aggregate is so dramatically increased that the solubility of the molecules is impaired. Aggregation is also known to potentially decrease the activity of the enzyme as compared to authentic oligomeric molecules.

The term “oligomer formation” as used herein refers to a reversible association of polypeptide chains whereby the ratio of oligomeric proteins and monomeric proteins at a certain protein concentration and at thermodynamic equilibrium is described by the association constant.

The term “non-aggregated” refers to polypeptides that are present either as oligomers or monomers or a mixture of oligomers and monomers at equilibrium according to the law of mass interaction.

The term “improved physicochemical properties” as used herein refers to a decrease in aggregate formation of polypeptides. For the purpose of definition, aggregate formation of the polypeptides of this invention is determined by quantitation of the fractions of a gelfiltration experiment in which aggregated PDE elutes, and of the fractions in which non-aggregated PDE elutes. The degree of aggregation is calculated in percent of non-aggregated PDE based on total (aggregated and non-aggregated) PDE. For the purpose of the definition of aggregation, aggregated PDE was considered to elute in fractions 2 to 4, in which aggregated molecules of at least 2 Mio Daltons eluted, and non-aggregated PDE was considered to elute in fractions 5 to 11, corresponding to the fractions in which molecules of about 0.3 Mio Daltons eluted, when the following conditions were used for gel filtration: 50 μl of Ni-NTA agarose purified isoform preparation were injected into a Superose 12 size exclusion column (type PC3.2/30; Amersham Pharmacia Biotech), equilibrated with 50 mM TrisHCl pH 7.7, 100 mM NaCl, 0.5 mM MgCl2 at a flow rate of 0.1 ml/min at 4° C. Chromatograms were recorded at 278 nm. Starting from the elution volume onwards, the column eluate was collected as 50 μl fractions.

The term “long form PDE” as used herein refers to PDE isoforms which comprise an isoform specific N-terminal domain, linker regions LR1 and/or LR2, and upstream conserved regions UCR1 and UCR2.

The term “UCR1 start” as used herein refers to the first amino acid of the UCR1 domain of PDE4D, as annotated in SEQ ID No. 1 to 7.

The term “LF1 site” as used herein refers to the first amino acid of the splice site of PDE4D as indicated in SEQ ID No. 1-7.

The term “amino acids upstream of LF1” refers to the position of an amino acid counted from the LF-1 site towards the amino terminus of the native protein.

The term “D3” as used herein refers to the PDE4D3 isoform. The term “D4” as used herein refers to the PDE4D4 isoform. The term “D5” as used herein refers to the PDE4D5 isoform. The term “D6” as used herein refers to the PDE4D6 isoform as provided by WO02/074992. The term “D7” as used herein refers to the PDE4D7. isoform as provided by WO02/074992. The term “D8” as used herein refers to the PDE4D8 isoform as provided by WO02/074992. The polypeptide sequences of the human PDE4D isoforms D3, D4, D5, D6, D7, and D8 are shown in SEQ ID No.1 and 3-7.

The term “Ser54” as used herein refers to the Ser residue in position 54 of PDE4D3, and to the Ser residue in the respective position of any of the other PDE polypeptides. The position corresponding to Ser54 of PDE4D3 is annotated in SEQ ID No. 1 to 7 for the different isoforms and the core sequence which is common to all the PDE4D isoforms.

The term “Ser579” as used herein refers to the Ser residue in position 579 of PDE4D3, and to the Ser residue in the respective position of any of the other PDE polypeptides. The position corresponding to Ser579 of PDE4D3 is annotated in SEQ ID No. 1 to 7 for the different isoforms and the core sequence which is common to all the PDE4D isoforms.

The term “tubulin association” as used herein refers to the tubulin content of purified PDE preparations.

As used herein, the term “agonist” also refers to activators, and the term “antagonist” also refers to inhibitors of PDE phosphodiesterase activity.

The disadvantage of native recombinant PDE polypeptides resides in the formation of aggregates of the polypeptides, which gives rise to sub-optimal conditions for the identification of modulators of PDE phosphodiesterase activity in screening assays since aggregate formation is known to lead to polypeptide precipitation and to potentially impair activity of a polypeptide. The present invention is directed to the generation of PDE polypeptides with improved physicochemical properties which are particularly useful for the purpose of identifying agonists or antagonists of these enzymes.

The problem of aggregate formation of recombinant PDE polypeptides was overcome in the present invention by generating a polypeptide comprising a PDE polypeptide sequence with the exception of the PDE4A polypeptide sequence, with an amino-terminal deletion, said polypeptide exhibiting decreased aggregate formation, as shown in FIGS. 2 and 3. These polypeptides had a specific activity that is comparable to the specific activity of the native recombinant proteins, as is shown in FIG. 1. In a preferred embodiment, the invention provides a polypeptide comprising a PDE polypeptide sequence, with an amino-terminal deletion wherein the proportion of non-aggregated PDE in the total protein preparation is from 55 %, preferably from 60%, more preferably from 65%, most perferably from 68% to 100%, preferably to 90%, more preferably to 80%, most preferably to 70% of total protein, as determined by the quantitation of eluted fractions from a gel filtration of the PDE fractions. Quantitation was performed as follows: Equal volume aliquots of fractions from Size Exclusion Chromatography, run as hereinbefore described, were analyzed by SDS-PAGE (one gel per run and isoform, FIG. 3). Optical densities of Coomassie stained PDE were integrated as follows: After electrophoresis the Coomassie stained polyacrylamide gel was imaged by a video imaging system (FIG. 1). Optical densities of PDE bands were integrated using a Macintosh computer and the public domain software “NIH Image”, version 1.61 (developed at the U.S. National Institutes of Health and available from NIH or on the NIH website at http://rsb.info.nih.gov/nih-image/). The integrated arbitrary units per PDE band as returned by the software reflect the relative PDE concentrations within the original pools. Integrated densities from PDE bands of fractions 2-4 were added up, giving a relative amount of aggregated PDE, PDE_{agg} (FIG. 3). These fractions contained molecules of at least 2 Mio Daltons. Similarly, integrated densities from PDE bands of fractions 5-11 were added up, giving a relative amount of non-aggregated PDE, PDE_{non-agg}. In fractions 5-11, molecules in the range of 0.3 Mio Daltons eluted. Relative amount of total PDE, PDEₜₒₜₐₗ, was given by the sum of PDE_{agg} and PDE_{non-agg}. The percentage of non-aggregated PDE was given by the ratio of PDE_{non-agg} and PDEₜₒₜₐₗ, multiplied by 100.

In a preferred embodiment of the present invention the PDE polypeptide sequence with an amino-terminal deletion is a long form PDE polypeptide sequence. In a more preferred embodiment, the PDE polypeptide sequence is a PDE4 polypeptide sequence, preferably a long form PDE4 polypeptide sequence. In an even more preferred embodiment, the polypeptide sequence is a PDE4D polypeptide sequence, preferably a long form PDE4D polypeptide sequence.

Methods of generating amino-terminal deletion mutants of polypeptides are well known. Such deletion mutants can be generated by using site directed mutagenesis, e.g. by using PCR (Sambrook et al., Molecular Cloning: A Laboratory Manual (1989), Cold Spring Harbor Laboratory Press, New York, USA).

In one embodiment of the present invention, the polypeptide comprises a PDE polypeptide sequence starting between 25 amino acids, preferably between 20 amino acids, more preferably between 15 amino acids, even more perferably between 10 amino acids, most preferably between 5 amino acids upstream of LF1 and the LF1 site.

In another embodiment the PDE polypeptide sequence starts at any amino acid located between the LF1 splice site and the first amino acid of the UCR1 start of the native PDE polypeptide. In the case of the most preferred embodiment of the PDE polypeptide, PDE4D, these sites are identical in all of the long form PDE4D isoforms.

In another more preferred embodiment of the present invention, the amino-terminal deletion is such that the PDE polypeptide sequence starts at the LF1 splice site of the native PDE polypeptide. This PDE polypeptide sequence is referred to as the “core construct”. The core construct spans a sequence of PDE which is shared by all isoforms. The core construct sequence of PDE4D is shown in SEQ ID No. 2.

In another more preferred embodiment of the present invention, the start of the PDE polypeptide sequence is located 13 amino acids upstream of the UCR1 start of the native PDE polypeptide. The location of this position in the long form PDE4D isoforms and the core construct of PDE4D is shown in SEQ ID No. 1-7.

The basis for the generation of the above mentioned PDE polypeptide with an amino-terminal deletion and improved physicochemical characteristics can be any isoform of PDE, with the exception of PDE4A. For the most preferred embodiment the PDE polypeptide sequence is an isoform of PDE4D selected from the group consisting of D3, D4, D5, D6, D7, and D8. However, the present invention also extends to further isoforms of PDE that have not yet been identified.

Also of use to the present invention are mutants of the PDE polypeptide herein before described. Of particular interest is a PDE polypeptide sequence comprising one or more mutations of Serine residues. Preferably, said Serines are mutated to either Alanine or Aspartic acid. In a most preferred embodiment, said Serine residues are selected from the group consisting of Ser54 and Ser579. Such Serines represent targets for phosphorylation of PDE polypeptides. Phosphorylation of some of these sites has been shown to activate or inhibit the phosphodiesterase activity of PDEs.

Methods of generating amino acid substitutions in polypeptides are well known to those skilled in the art of Molecular Biology, e.g. methods such as site directed mutagenesis as described in Sambrook et al., Molecular Cloning: A Laboratory Manual (1989), Cold Spring Harbor Laboratory Press, New York, USA.

In another preferred embodiment of the present invention, the PDE polypeptide hereinbefore described exhibits decreased tubulin association, thus allowing to obtain purer PDE polypeptide preparations. To determine the decrease in tubulin association, the ratio of PDE molecules per tubulin molecules in the preparation of PDE polypeptide was calculated by quantitation of SDS-PAGE. Preferably, the ratio of PDE molecules per tubulin molecules is at least 5, preferably at least 10, more preferably at least 15, even more preferably at least 20, most preferably at least 25 molecules of PDE per molecules of tubulin. The tubulin content of PDE preparations was determined as follows: Equal volume aliquots of fractions from SEC were analyzed by SDS-PAGE (one gel per run and isoform, FIG. 3). Optical densities of Coomassie stained PDE and tubulin bands were integrated as described hereinbefore for PDE aggregation quantitation. Integrated densities from PDE bands of fractions 2-11 were added up, giving a relative amount of total PDE, PDEₜₒₜₐₗ. Likewise, integrated densities from tubulin bands of fractions 2-11 were added up, giving tubulin₂₋₁₁, and thereby the relative amount of total tubulin, tubulinₜₒₜₐₗ. The ratio of PDE and tubulin (FIG. 4C) was identical with the ratio of the relative amount of total PDE4D, PDEₜₒₜ, and relative amount of total tubulin, tubulintotal, on a mass per mass basis.

The PDE polypeptides from which the peptide of this invention is generated by amino-terminal deletion as hereinbefore described include polypeptides that are substantially homologous or identical to the polypeptides shown in SEQ ID No. 1-7 and have, in their native form, the phosphodiesterase activity and specificity of PDE. In particular, these PDE polypeptides also include the previously published polypeptide sequences of PDE4D3 to D8 as defined in U50159 (D3), L20969 (D4), S:1059276 (D5) and the sequences for D6, D7 and D8 as disclosed in WO02/074992. As used herein, two polypeptides (or a region of the polypeptides) are substantially homologous or identical when the amino acid sequences are at least 45-55%, typically at least 70-75%, more typically at least 80-85%, and most typically greater than 90% or more homologous or identical. To determine the percent homology or identity of two amino acid sequences, the sequences are aligned for optimal comparison purposes (e.g., gaps can be introduced in the sequence of one polypeptide for optimal alignment with the other polypeptide or nucleic acid molecule). The amino acid residues or nucleotides at corresponding amino acid positions are then compared. When a position in one sequence is occupied by the same amino acid residue as the corresponding position in the other sequence, then the molecules are homologous at that position. As used herein, amino acid “homology” is equivalent to amino acid “identity”. The percent homology between the two sequences is a function of the number of identical positions shared by the sequences (i.e., percent homology equals the number of identical positions/total number of positions times 100). The PDE polypeptide sequences of the present invention are preferably mammalian, most preferably human. The present invention also includes variants of the polypeptides hereinbefore described with deletions, insertions, inversions, repeats, and substitutions selected according to general rules known in the art so as to have little effect on activity. For example, guidance concerning how to make phenotypically silent amino acid substitutions is provided in Bowie, Science 247 (1990), 1306-1310, wherein the authors indicate that there are two main strategies for studying the tolerance of an amino acid sequence to change.

The present invention provides polynucleotides that comprise polynucleotide sequences that encode the PDE polypeptide sequences hereinbefore described. As an example for a sequence comprised in such a polynucleotide, the polynucleotide sequence encoding the “core construct” of PDE4D is shown in SEQ ID No. 8. However, the present invention relates to all polynucleotides comprising polynucleotide sequences encoding any of the PDE polypeptide sequences hereinbefore described.

The present invention provides an expression vector or virus comprising a polynucleotide sequence that encodes the PDE polypeptide sequences hereinbefore described and which is capable of directing expression of the polynucleotide sequence in a compatible prokaryotic or eukaryotic host cell. The invention also provides a prokaryotic or eukaryotic host cell transformed or infected with such an expression vector or virus. The invention further provides a process for the production of such a polypeptide comprising culturing the host cell expressing the PDE polypeptide sequences hereinbefore described in a suitable medium so that said polypeptide is expressed, and purifying said polypeptide from the cells. In a preferred embodiment, the virus used for the expression of a modified PDE polypeptide is a recombinant baculovirus. Methods of generating recombinant baculoviruses for expression of recombinant proteins are well known to the man skilled in the art. Preferably, the recombinant baculovirus is based on pFASTBac as the transfer vector. The host cell of a preferred embodiment of the invention is an insect cell. Insect cells that can be infected with recombinant baculoviruses for the purpose of expressing recombinant proteins are well known in the art. The preferred process for the production of a modified PDE polypeptide is a process comprising culturing the insect cell expressing the PDE polypeptide sequences hereinbefore described in a suitable medium so that said polypeptide is expressed, and purifying said polypeptide from the insect cell. To facilitate purification, the PDE polypeptide may also comprise a tag. Different types of such tags and the methods to join them with polynucleotide sequences encoding a polypeptide are well known to the person skilled in the art, e.g. myc-tag, FLAG-tag, six Histidine tags (hereinafter referred to as 6×His tag). A review on tagging of proteins can be found e.g. in Fritze and Anderson, Methods in Enzymology (2000) 327, 3-16. It is well known that such tags may be joined to either side of the polypeptide. Some tags are known which may also be removed enzymatically from the purified polypeptide, e.g. FLAG-tag fused to the amino-terminus of a protein may be removed by digestion with bovine enterokinase (Hopp, T. P.(1988) Biotechnology, 6, 1204-1210). In a preferred embodiment the PDE polypeptides hereinbefore described are linked at their C-terminus to a 6×His tag. The use of such 6×His tags for ease of purification of recombinant polypeptides, and methods of purification of 6×His tagged polypeptides are well known (e.g. in Sisk et al. (1994) J. Virol. 68, 766, and Petty, K. J. (1996) in: Ausubel, F. M. et al., eds. Current Protocols in Molecular Biology, Vol. 2, New York: John Wiley and Sons).

In a preferred embodiment, the polypeptide of the present invention is purified. As used herein, a polypeptide is said to be “purified” when it is substantially free of cellular material when it is isolated from recombinant and non-recombinant cells. A polypeptide, however, can be joined to another polypeptide with which it is not normally associated in a cell (e.g. in a “fusion protein”) and still be “purified”. Preferably, the polypeptides of the present invention are purified. The polypeptides of the invention can be purified to homogeneity. It is understood, however, that preparations in which the polypeptide is not purified to homogeneity are useful. The critical feature is that the preparation allows for the desired function of the polypeptide, even in the presence of considerable amounts of other components. Thus, the invention encompasses various degrees of purity. In one embodiment, the language “substantially free of cellular material” includes preparations of the polypeptide having less than about 30% (by dry weight) other proteins (i.e., contaminating protein), less than about 20% other proteins, less than about 10% other proteins, or less than about 5% other proteins. When a polypeptide is recombinantly produced, it can also be substantially free of culture medium, i.e., culture medium represents less than about 20%, less than about 10%, or less than about 5% of the volume of the polypeptide preparation.

The invention further provides an antibody against any polypeptide hereinbefore described. Preferably, the antibody binds to an epitope of the N-terminal domain of the PDE polypeptides. In another preferred embodiment the antibody binds to the N-terminus of a PDE4D polypeptide selected from the group consisting of D3, D4, D5, D6, D7, and D8. Such antibodies may be useful for different purposes. They may be used for detection of the polypeptides of this invention. They may also be used for affinity purification of the polypeptides of the present invention. Furthermore, they may be used in a screening assay to identify agonists or antagonists of PDE4D phosphodiesterase activity.

The present invention also provides for a use of the polypeptides hereinbefore described for identifying an agonist or antagonist of phosphodiesterase activity.

The polypeptides of the present invention can be used for a process of identifying and obtaining a drug candidate for therapy of a vascular disorder, said process comprising measuring the activation or inhibition of the phosphodiesterase activity of any of the above mentioned polypeptides. Different methods for measuring phosphodiesterase activity are known, e.g. from Owens et al., Biochem. J. (1997) 326, 53-60. Such a drug candidate may be a compound that is either an agonist or an antagonist of the phosphodiesterase activity of PDE.

The present invention also provides a screening assay for the identification of agonists or antagonists of phosphodiesterase activity comprising at least one of the above described polypeptides. In addition, the polypeptides hereinbefore described can be used for crystallization. Crystals of the polypeptides are useful for assaying the interaction of compounds with phosphodiesterases. Thus, the present invention also provides a method of assaying a candidate agonist or antagonist for. its ability to interact with a phosphodiesterase comprising crystallizing the polypeptide hereinbefore described in a condition suitable for X-ray crystallography and conducting said X-ray crystallography on said polypeptide.

The present invention also provides compounds identified by any of the processes or methods hereinbefore described.

The present invention provides a pharmaceutical composition comprising an activator or inhibitor of the phosphodiesterase activity of the PDE polypeptide of the present invention, and a pharmaceutically acceptable carrier. Any conventional carrier material can be utilized. The carrier material can be an organic or inorganic one suitable for eteral, percutaneous or parenteral administration. Suitable carriers include water, gelatin, gum arabic, lactose, starch, magnesium stearate, talc, vegetable oils, polyalkylene-glycols, petroleum jelly and the like. Furthermore, the pharmaceutical preparations may contain other pharmaceutically active agents. Additional additives such as flavoring agents, stabilizers, emulsifying agents, buffers and the like may be added in accordance with accepted practices of pharmaceutical compounding.

An agonist or antagonist of the phosphodiesterase activity of the PDE polypeptide of the present invention or a pharmaceutically acceptable salt thereof may be used for the preparation of a medicament for the treatment of diseases caused by elevated or reduced phosphodiesterase activities. Such diseases include asthma, a topic dermatitis, depression, septic shock, toxic shock, autoimmune diabetes, AIDS, Crohn's disease, multiple sclerosis, psoriasis, allograft rejection, ulcerative colitis, cachexia, cerebral malaria, allergic rhinoconjunctivitis, osteoarthritis, rheutmatoid arthritis, autoimmune encephalomyelitis, and vascular diseases such as reperfusion injury, cerebral ischemia, restenosis, stroke or peripheral arterial occlusive disease. The invention is, however, not limited to the diseases listed hereinbefore, but extends to all diseases that depend on elevated or reduced phosphodiesterase activity. Of particular interest to this invention is the use of such an agonist or antagonist of phosphodiesterase activity of PDE or a pharmaceutically acceptable salt thereof for the preparation of a medicament for the treatment of a vascular disease. The most preferred embodiment is the use of such agonist or antagonist of the phosphodiesterase activity of PDE or a pharmaceutically acceptable salt thereof for the preparation of a medicament for the treatment of stroke.

Furthermore, the present invention provides a kit for identifying agonists or antagonists of phosphodiesterase activity comprising at least one of the polypeptides hereinbefore described.

In the present invention, the products, methods, processes, assays and uses substantially as herein before described, especially with reference to the foregoing examples, are also claimed.

The invention will be further described by the following non-limiting examples.

### EXAMPLES

In the following the term “isoform” relates to the natural PDE4D splice variants D3 and D5-D8 and the invented core variant, DC.

Cloning of the PDE4D Isoforms 3-8 and the Core Construct

Core Construct:

A cDNA encoding the core fragment that is common for all the PDE4D isoforms 3-8 starting with the amino acid sequence FDV carboxyterminal to the LF1 splice site was generated by PCR using a 5′ oligonucleotide with a HindIII cloning site (gatgaattcaagctttttgatgtggacaatggcaca; SEQ ID NO:9) introducing two additional amino acids (K and L) in front of the FDV sequence. At the 3′ end a set of primers was used that either generated the native sequence (gtgatatctcattatcacgtgtcaggagaacgatcatctatgaca; SEQ ID NO: 10) or added a sequence encoding 6×His residues (gtgatatctcattatcaa tgggatggtgatggtgcgtgtcaggagaacgatcatctatgac; SEQ ID NO: 11) to enable rapid purification of the recombinant proteins. The cDNA encoding the core construct was cloned as a EcoRI-EcoRV fragment into the expression vector pENTR™ 1a (GIBCO/BRL)

Isoforms (Except Core Construct):

The DNA fragments encoding the isoform specific N-termini were generated by using synthetic oligonucleotides with terminal restriction enzyme sites for EcoRI and HindIII incorporated for directional cloning. These isoform specific DNA fragments were fused to the core construct sequence via the HindIII site introducing two additional amino acid residues (K and L). The integrity of the clones was confirmed by DNA sequencing prior to expression.

Expression of the PDE4D Isoforms and the Core Construct in Insect Cells

The cDNAs were cloned into the pFASTBAC1 vector (Life Technologies. Inc) for expression in insect cells, and the products were confirmed by sequencing. After recombination into the baculovirus genome the purified viral DNAs were transformed into the insect cells. Sf9 cells were cultured at 27° C. in TC100 medium (BioWhittaker) with 5% (v/v) fetal calf serum. Virus stocks were generated with a titer of 1.5×10⁹ pfu/ml. For large scale production of the isoforms 1-24 L fermentations of Sf9 cells were infected with a MOI of 1.

In one example, the 6×His tagged PDE4D polypeptides DC, D3, D5, D6, D7 and D8 were produced in Sf9 cells in 1 L spinner flasks using SF1 medium in the absence of serum. Infected cells were harvested 3 days after infection with the recombinant baculoviruses.

In another example, the PDE4D core construct DC was produced in a 24 L Airlifter Fermenter with 15 L medium (SF1 with 0% serum), 0.15 L lipids and 9 L Sf9 cells. During the entire fermentation procedure the cells were cultured at pH 6.2, 27.0±0.2° C. and a pO2 of 30.0±0.5%. Cells were grown for 3 days. The cell number at infection was 2.3×10⁶ cells/ml. Cells were infected with 450 ml recombinant baculovirus. Cells were harvested at 68 h post infection and the cell pellet as well as the concentrated supernatant stored at −80° C. until further processing.

Purification of 6×His Tagged PDE4D Isoforms

Sf9 cells from 1 liter culture broth, overexpressing the respective isoform, were resuspended on ice in 50 ml 50 mM HEPES pH 7.8, 300 mM NaCl 10 mM imidazole, 1 mM DTT, supplemented with protease inhibitors (one protease inhibitor cocktail tablet “complete, EDTA-free”; Roche). Opening of cells was performed by use of a 50 ml Dounce homogenizator and the homogeneous mixture was centrifuged for 1 hour at 70'000 g and 4° C. (Kontron TFT 45.94 rotor at 30'000 rpm). The supernate was filtrated through a filter with a pore size of 1.2 μM (Minisart; Sartorius, Germany) and then applied to a 6 ml Ni-NTA agarose column at 2 ml/min. After equilibration with 50 mM HEPES pH 7.8, 300 mM NaCl, 10 mM imidazole, protein was eluted with a linear 30 ml gradient from 10 to 230 mM imidazole in the same buffer. Fractions containing the PDE4D isoform as analyzed by Coomassie stained SDS-PAGE were pooled and stored frozen at −80° C. Fresh Ni-NTA agarose material was used for every different PDE4D isoform preparation in order to prevent cross contamination of isoforms.

Specific Activities of 6×His Tagged PDE4D Isoforms

Relative concentrations of Ni-NTA agarose purified isoform preparations were estimated by SDS-PAGE. Equal volume amounts of isoform preparations were applied to a gradient gel (4-12% NuPage; Invitrogen). After electrophoresis the Coomassie stained polyacrylamide gel was imaged by a video imaging system (FIG. 1). Optical densities of PDE4D bands were integrated using a Macintosh computer and the public domain software “NIH Image”, version 1.61 (developed at the U.S. National Institutes of Health and available from NIH or on the NIH website at http://rsb.info.nih.gov/nih-image/). The integrated arbitrary units per PDE4D band as returned by the software reflect the relative PDE4D concentrations within the original pools (FIG. 1, “amount”). Identities of PDE4D and tubulin bands had been verified by independent SDS-PAGE, excision of corresponding bands, trypsin cleavage and identification of tryptic peptides by MALDI-MS.

Activities of equal volume amounts of 10⁶-fold diluted purified isoforms were determined by use of a commercial radioactive phosphodiesterase assay (cAMP-dependent phosphodiesterase [³H] assay; Amersham Pharmacia Biotech), following the instructions of the manufacturer. The obtained arbitrary activity units reflect the relative PDE4D activities within the original pools (FIG. 1, “activity”).

Relative specific activities of PDE4D isoforms were calculated by dividing relative activity values by relative concentration values (FIG. 1, “specific activity”).

Qualitative Investigation of Aggregation by Size Exclusion Chromatography (SEC)

50 μl of Ni-NTA agarose purified isoform preparation was injected into a Superose 12 size exclusion column (type PC3.2/30; Amersham Pharmacia Biotech), equilibrated with 50 mM TrisHCl pH 7.7, 100 mM NaCl, 0.5 mM MgCl2 at a flow rate of 0.1 ml/min at 4° C. Chromatograms were recorded at 278 nm. Starting from the elution volume, the column eluate was collected as 50 μl fractions (FIG. 2).

Quantification of Aggregates and Tubulin Content

Equal volume aliquots of fractions from SEC were analyzed by SDS-PAGE (one gel per run and isoform, FIG. 3). Optical densities of Coomassie stained PDE4D and tubulin bands were integrated as described above. Integrated densities from PDE4D bands of fractions 2-4 were added up, giving a relative amount of aggregated PDE4D, PDE_{agg} (FIG. 3). Similarly, integrated densities from PDE4D bands of fractions 5-11 were added up, giving a relative amount of non-aggregated PDE4D, PDE_{non-agg}. Relative amount of total PDE4D, PDEₜₒₜₐₗ, is given by the sum of PDE_{agg} and PDE_{non-agg}. The percentage of non-aggregated PDE is given by the ratio of PDE_{non-agg} and PDEₜₒₜₐₗ, multiplied by 100 (FIG. 4B).

Similarly, integrated densities from tubulin bands of fractions 2-11 were added up, giving tubulin₂₋₁₁, and thereby the relative amount of total tubulin, tubulinₜₒₜₐₗ. The ratio of PDE and tubulin (FIG. 4C) is identical with the ratio of relative amount of total PDE4D, PDEₜₒₜ, and relative amount of total tubulin, tubulintₜₒₜₐₗ.

Activity Assay and Inhibition of Phosphodiesterase Activity

An IMAP FP-Assay was used for the determination of phosphodiesterase activity. The phosphodiesterase activity of the core contruct and PDE4D3 was measured using the HEFP Phosphodiesterase Assay Kit (Molecular Devices). 2 μl of PDE4D3 or PDE4D core construct, 2 μl of cAMP (to a final concentration of 40 nM) and 1 μl of test substance or carrier were incubated for 45 min on a shaker. 12 μl of Binding Solution provided by the kit (with beads diluted 1:320) were added, and the reaction mixture incubated on a shaker for 2 hours. Fluorescence polarisation of the samples was measured in a Packard BioScience Fusion a-FP HT using as an emission filter a Polarizer 535, and as an excitation filter, Fluorescein 485/20. Inhibition of the phosphodiesterase activity of the PDE4D core construct was determined using Rolipram as inhibitor, and using PDE4D core construct at 30 ng/ml.

1. A polypeptide comprising a PDE polypeptide sequence with an amino-terminal deletion, said polypeptide exhibiting decreased aggregate formation.

2. The polypeptide of claim 1, wherein said PDE polypeptide sequence is a long form PDE polypeptide sequence.

3. The polypeptide of claim 1, wherein said PDE polypeptide sequence is a PDE4 polypeptide sequence.

4. The polypeptide of claim 3 wherein said PDE polypeptide sequence is a PDE4D polypeptide sequence.

5. The polypeptide of claim 4 wherein the proportion of non-aggregated polypeptide in the total polypeptide is from 55% to 100% of total polypeptide.

6. The polypeptide of claim 1 wherein the proportion of non-aggregated polypeptide in the total polypeptide is from 55% to 100% of total polypeptide.

7. The polypeptide of claim 1, wherein the polypeptide sequence starts at any amino acid located between the LF 1 splice site and the first amino acid of the UCRI start of the native PDE polypeptide.

8. The polypeptide of claim 7, wherein the polypeptide sequence starts at the LF1 splice site of the native PDE polypeptide.

9. The polypeptide of claim 7, wherein the PDE polypeptide sequence start is located 13 amino acids upstream of the UCRI start of the native PDE polypeptide.

10. The polypeptide of claim 4 wherein the PDE4D polypeptide sequence is a sequence of an isoform selected from the group consisting of D3, D4, D5, D6, D7, and D8.

11. The polypeptide of claim 1 wherein said polypeptide comprises one or more mutations of Serine residues.

12. The polypeptide of claim 11 wherein said Serine residues are mutated to either Alanine or Aspartic acid.

13. The polypeptide of claim 11 wherein said Serine residues are selected from the group consisting of Ser54 and Ser579.

14. The polypeptide of claim 1 wherein said polypeptide exhibits decreased tubulin association.

15. A polynucleotide sequence encoding a polypeptide of claim 1.

16. A polynucleotide sequence encoding a polypeptide of claim 2.

17. A polynucleotide sequence encoding a polypeptide of claim 3.

18. A polynucleotide sequence encoding a polypeptide of claim 4.

19. A polynucleotide sequence encoding a polypeptide of claim 5.

20. A polynucleotide sequence encoding a polypeptide of claim 6.

21. A polynucleotide sequence encoding a polypeptide of claim 7.

22. A polynucleotide sequence encoding a polypeptide of claim 8.

23. A polynucleotide sequence encoding a polypeptide of claim 9.

24. A polynucleotide sequence encoding a polypeptide of claim 10.

25. An expression vector or virus comprising a polynucleotide sequence as claimed in claim 15 capable of directing expression of the polynucleotide sequence in a compatible prokaryotic or eukaryotic host cell.

26. A virus as claimed in claim 25 wherein the virus is a recombinant baculovirus.

27. A prokaryotic or eukaryotic host cell transformed or infected with the expression vector or virus of claim 25.

28. A host cell of claim 27, wherein the host cell is an insect cell.

29. A prokaryotic or eukaryotic host cell transformed or infected with the expression vector or virus of claim 26.

30. A host cell of claim 29, wherein the host cell is an insect cell.

31. A process for the production of a polypeptide of claim 1, comprising culturing the host cell of claim 29 in a suitable medium so that said polypeptide is expressed, and purifying said polypeptide from the cells.

32. A process for the production of a polypeptide of claim 31 wherein the host cell is an insect cell.

33. A process for the production of a polypeptide of claim 1 comprising culturing the host cell of claim 27 in a suitable medium so that said polypeptide is expressed, and purifying said polypeptide from the cells.

34. A process for the production of a polypeptide of claim 33 wherein the host cell is an insect cell.

35. An antibody against a polypeptide of claim 1.

36. An antibody of claim 35, wherein the antibody binds to an epitope of the N-terminal domain of the polypeptide of claim 1.

37. A screening assay for the identification of agonists or antagonists of phosphodiesterase activity comprising at least one polypeptide of claim 1.

38. A process for identifying and obtaining a drug candidate for therapy of an inflammatory disease, said process comprising measuring the activation or inhibition of the phosphodiesterase activity of a polypeptide of claim 1.

39. A compound identified according to the assay of claim 37.

40. A compound identified according to the process of claim 38.

41. A pharmaceutical composition comprising an agonist or antagonist of the phosphodiesterase activity of the polypeptide of claim 1, and a pharmaceutically acceptable. carrier.

42. A method of assaying a candidate agonist or antagonist for its ability to interact with a phosphodiesterase comprising crystallizing a polypeptide of claim 1 in a condition suitable for X-ray crystallography and conducting said X-ray crystallography on said polypeptide.

43. A crystallized polypeptide of claim 1.
